(19) United States
(12) Patent Application Publication
(10) Pub. No.: US 2019/0000422 A1
(43) Pub. Date: Jan. 3, 2019

(54) ELASTICITY IMAGING IN HIGH INTENSITY FOCUSED ULTRASOUND

(71) Applicant: Butterfly Network, Inc., Guilford, CT (US)

(72) Inventors: Lawrence C. West, San Jose, CA (US); Kailiang Chen, Branford, CT (US); Tyler S. Ralston, Clinton, CT (US); Sarp Satir, Guilford, CT (US); Jaime Scott Zahorian, Guilford, CT (US)

(21) Appl. No.: 16/022,841

(22) Filed: Jun. 29, 2018

Related U.S. Application Data
(60) Provisional application No. 62/567,660, filed on Oct. 3, 2017.
(60) Provisional application No. 62/527,281, filed on Jun. 30, 2017.

(51) Int. Cl. A61B 8/08; G01S 15/89; G01S 7/52

(52) U.S. Cl. CPC A61B 8/485; A61N 2007/0043; G01S 7/52042; G01S 15/8984

(57) ABSTRACT

Ultrasound devices configured to perform high-intensity focused ultrasound (HIFU) are described. An ultrasound device may include HIFU units configured to emit high acoustic intensities and elasticity detectors configured to determine characteristics of the target area of a human body based on the elasticity of the target area. The elasticity detectors may determine, e.g., whether the target area is healthy, and if not, the type cell in need of treatment (e.g., the type of cancer cell present in the target area). In one example, the elasticity detectors may be configured to determine the stiffness of the target area, which may provide an indication as to the type of cell present in the area, by estimating the velocity of a shear wave propagating away from the target area. The shear wave may arise in response to the application of an ultrasound wave to the target area.

### CROSS-REFERENCE TO RELATED APPLICATIONS

This application claims the benefit under 35 U.S.C. § 119(e) of U.S. Provisional Patent Application Ser. No. 62/527,281, entitled “SHEAR WAVE IMAGING IN HIGH INTENSITY FOCUSED ULTRASOUND,” filed on Jun. 30, 2017 under Attorney Docket No. B1348.70054US00, which is hereby incorporated herein by reference in its entirety.

This application claims the benefit under 35 U.S.C. § 119(e) of U.S. Provisional Patent Application Ser. No. 62/567,660, entitled “ELASTICITY IMAGING IN HIGH INTENSITY FOCUSED ULTRASOUND,” filed on Oct. 3, 2017 under Attorney Docket No. B1348.70054US01, which is hereby incorporated herein by reference in its entirety.

### TECHNICAL FIELD

The present application relates to ultrasound devices.

### BACKGROUND

High intensity focused ultrasound (HIFU) is used in some medical procedures to kill cancer cells with high frequency sound waves. These waves deliver a strong beam to a specific part of a cancer. Some cells die when this high intensity ultrasound beam is focused directly onto them.

### SUMMARY

Some embodiments relate to an apparatus comprising one or more high intensity focused ultrasound (HIFU) units configured to generate HIFU waves and one or more elasticity detectors configured to sense a characteristic of a shear wave, the one or more HIFU units and the one or more elasticity detectors being disposed on a common ultrasound device.

In some embodiments, the ultrasound device comprises a substrate on which ultrasonic transducers are integrated.

In some embodiments, the ultrasound device comprises a handheld probe.

In some embodiments, at least one of the one or more HIFU units comprises a capacitive micromachined ultrasound transducer (CMUT).

In some embodiments, at least one of the one or more HIFU units is configured to emit an acoustic intensity that is between 500 W/cm² and 20 KW/cm².

In some embodiments, the one or more HIFU units are disposed on a first substrate, the first substrate being bonded to a second substrate comprising electronic circuitry electrically coupled to the one or more HIFU units.

In some embodiments, the one or more elasticity detectors are configured to sense a velocity of the shear wave.

Some embodiments relate to a method for treating medical conditions. The method may comprise applying an acoustic wave on a portion of a tissue of a human body, identifying one or more cells in need of treatment by sensing a characteristic of a shear wave arising in response to the application of the acoustic wave and propagating away from the portion of the tissue; and applying an HIFU wave on the one or more cells in need of treatment.

In some embodiments, the method may further comprise monitoring a state of the one or more cells in need of treatment by sensing stiffness variations in the one or more cells in need of treatment.

In some embodiments, the acoustic wave is a first acoustic wave and the shear wave is a first shear wave, and wherein sensing stiffness variations in the one or more cells in need of treatment comprises: applying a second acoustic wave to the one or more cells in need of treatment; and sensing a characteristic of a second shear wave arising in response to the application of the second acoustic wave and propagating away from the one or more cells in need of treatment.

In some embodiments, identifying one or more cells in need of treatment by sensing a characteristic of a shear wave propagating away from the portion of the tissue comprises identifying one or more cells in need of treatment by sensing a velocity of a shear wave propagating away from the portion of the tissue.

In some embodiments, applying an HIFU wave on the one or more cells in need of treatment comprises emitting an acoustic intensity that is between 500 W/cm² and 20 KW/cm².

In some embodiments, the method further comprises performing micro-cavitation on the one or more cells in need of treatment, wherein applying an HIFU wave on the one or more cells in need of treatment comprises applying an HIFU wave on the micro-cavitation.

In some embodiments, the method further comprises determining a state of the micro-cavitation by sensing a backscattered ultrasound wave.

In some embodiments, the method further comprises identifying a type of the tissue in need of treatment based on the characteristic of the shear wave

Some embodiments relate to a HIFU-on-a-chip device, comprising an arrangement of micro ultrasonic transducers integrated on a substrate and coupled to electronic circuitry configured to drive the arrangement of micro ultrasonic transducers to perform elasticity imaging and high intensity focused ultrasound (HIFU).

In some embodiments, the electronic circuitry is integrated on the substrate.

In some embodiments, the substrate is a first substrate, and wherein at least some of the electronic circuitry is disposed on a second substrate.

In some embodiments, the electronic circuitry comprises analog circuitry disposed on the first substrate and digital circuitry disposed on the second substrate.

In some embodiments, elasticity imaging comprises shear wave imaging.

Some embodiments relate to an apparatus comprising one or more high intensity focused ultrasound (HIFU) elements configured to provide HIFU and to generate shear waves.

Some embodiments relate to a method for treating medical conditions, the method comprising: performing micro-cavitation on a tissue of a subject by providing HIFU to the tissue; determining a state of the micro-cavitation; and performing an HIFU treatment by providing HIFU to the tissue.

In some embodiments, determining a state of the micro-cavitation comprises sensing a backscattered ultrasound wave.

In some embodiments, monitoring the state of the micro-cavitation once the HIFU treatment has been at least partially performed.

In some embodiments, the method further comprising identifying a presence of a tissue in need of treatment using elasticity imaging.

In some embodiments, the method further comprising identifying a type of a tissue in need of treatment using elasticity imaging.

In some embodiments, providing the HIFU to the tissue and determining the state of the micro-cavitation are performed using a common ultrasound device.

In some embodiments, providing the HIFU to the tissue comprises emitting ultrasound waves towards the tissue with a plurality of ultrasound devices.

Some embodiments relate to a method, comprising: emitting a first ultrasound signal toward at least one target area; and generating, based on a shear wave generated by the first ultrasound signal, a second ultrasound signal for treatment of at least a portion of the target area.

In some embodiments, the first and/or second ultrasound signals are generated using one or more ultrasound elements.

In some embodiments, the one or more ultrasound elements include at least one of the following: a capacitive micromachined ultrasound transducer (CMUT), piezoelectric transducer, lead zirconate titanate (PZT) element, lead magnesium niobate-lead titanate (PMN-PT) element, polyvinylidene difluoride (PVDF) element, high power ceramic element, PZT-4 ceramic element, and any combination thereof.

In some embodiments, the first and/or second ultrasound signals include at least one of the following: a high-intensity focused ultrasound (HIFU) signal, a non-HIFU ultrasound signal, and any combination thereof.

Some embodiments relate to a method for treating medical conditions, comprising: applying a high intensity focused ultrasound (HIFU) wave on a portion of a tissue of a human body, determining a state of the portion of the tissue by monitoring the portion of the tissue using ultrasound waves; and updating the application of the HIFU wave based on the determined state of the portion of the tissue.

In some embodiments, monitoring the portion of the tissue comprises monitoring a shear wave propagating through the tissue.

In some embodiments, monitoring the portion of the tissue further comprises estimating a velocity of the shear wave.

In some embodiments, monitoring the portion of the tissue further comprises estimating an elasticity of the portion of the tissue based on the estimated velocity of the shear wave and generating an elasticity map.

In some embodiments, the method further comprising identifying a region in need of treatment based on the elasticity map, and applying the HIFU wave to the region in need of treatment.

In some embodiments, monitoring the portion of the tissue comprises sensing a backscattered ultrasound wave.

In some embodiments, monitoring the portion of the tissue comprises comparing a first elasticity map of the tissue obtained before the application of the HIFU wave with a second elasticity map of the tissue obtained after the application of the HIFU wave.

In some embodiments, monitoring the portion of the tissue comprises monitoring a cross correlation or Doppler signal at a point of HIFU application.

In some embodiments, monitoring the portion of the tissue comprises monitoring a cross correlation or Doppler signal at a point of HIFU application, when the HIFU application is modulated at a lower frequency so as to create changes in particle motion as affected by elasticity in the tissue.

### BRIEF DESCRIPTION OF THE DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1A is a block diagram illustrating a system having an high intensity focused ultrasound (HIFU) unit and an elasticity detector, according to some non-limiting embodiments.

FIG. 1B is a schematic diagram illustrating an ultrasound device having a plurality of ultrasound elements arranged as a two-dimensional array, according to some non-limiting embodiments.

FIG. 1C is a schematic illustration of an ultrasound device having an ultrasonic transducer substrate bonded with an integrated circuit substrate, according to some non-limiting embodiments.

FIG. 1D is a schematic diagram illustrating a first handheld probe having a HIFU unit and a second handheld probe having an elasticity detector, according to some non-limiting embodiments.

FIG. 2A is a schematic diagram illustrating a system while emitting an ultrasound wave towards a tissue, according to some non-limiting embodiments.

FIG. 2B is a schematic diagram illustrating a shear wave generated in response to the ultrasound wave of FIG. 2A, according to some non-limiting embodiments.

FIG. 2C is a schematic diagram illustrating application of a HIFU wave to a tissue, according to some non-limiting embodiments.

FIG. 3 is a flowchart illustrating a method for treating medical conditions, according to some non-limiting embodiments.

FIG. 4 is a table illustrating elastic and velocity ranges for different medical conditions, according to some non-limiting embodiments.

### DETAILED DESCRIPTION

Some aspects of the present application provide a high intensity focused ultrasound (HIFU) system that utilizes elasticity imaging in conjunction with application of HIFU, wherein the elasticity imaging is performed using one or more ultrasound probes. One example of elasticity imaging is shear wave imaging, in which the elasticity of a tissue may be inferred based on the velocity or other characteristic of a shear wave propagating through a tissue. It should be appreciated, however, that other types of imaging based on elasticity of a tissue may be used.

Applicant has appreciated that the ability to use HIFU to treat medical conditions may be improved by employing shear waves for targeting the region being treated with HIFU and/or for verifying the HIFU treatment. HIFU is a therapeutic technology in which focused ultrasound energy is used to generate highly localized heating, cavitation, drug activation, or other treatments. HIFU may be applied, for example, to treat human tissues, for instance targeting cancers, cataracts, kidney stones, or other diseases. The stages of a HIFU procedure may include: (1) targeting the area at which to apply HIFU; (2) HIFU application; (3) verification of the HIFU application; and (4) verification that no healthy areas have been accidentally damaged. Some aspects of the present application utilize elasticity imaging as part of the targeting and/or verification stages. In some embodiments, the elasticity imaging is performed using the same ultrasound probe(s) used to apply the HIFU. Some aspects of the present application are directed to therapeutic systems comprising one or more units capable of providing HIFU treatment and one or more units arranged to perform elasticity imaging. The HIFU unit(s) and the elasticity detector(s) may be disposed on the same ultrasound device, such as in the same substrate (e.g., a silicon substrate), in the same support (e.g., a printed circuit board), or in the same housing (e.g., a handheld probe). Alternatively, the HIFU unit(s) and the elasticity detector(s) may be disposed on separate ultrasound devices, and the ultrasound devices may be arranged as an array. For example, a HIFU unit may be disposed in a first ultrasound device and a elasticity detector may be disposed on a second ultrasound device. In some embodiments, multiple HIFU units and/or multiple elasticity detectors may be used. In the embodiments comprising multiple HIFU units, the HIFU units may be disposed on the same ultrasound device, or may form an array of ultrasound devices. In the embodiments comprising multiple elasticity detectors, the elasticity detectors may be disposed on the same ultrasound device, or may form an array of ultrasound devices.

Applicant has appreciated that the use of HIFU for the treatment of medical conditions faces a few challenges. First, since the ultrasound waves used in HIFU are highly focused, it may be important in some embodiments to accurately direct such waves towards the tissues in need of treatment, and not toward unintended areas. However, identifying the location of these tissues with precision is often challenging due to the lack of reliable imaging techniques. Second, even when HIFU treatment is performed on tissue, it is difficult to determine whether the treatment has produced the desired results.

Elasticity imaging techniques of the types described herein may be configured to image portions of a human body or other subject by sensing a characteristic of the tissues (e.g., elasticity, stiffness, Young's modulus, pressure measured for example in pounds per square inch (PSI) or pascals, ratio of stress to strain, or other related quantities) or other target materials. In some embodiments, a perturbation (ultrasound, mechanical or any other suitable perturbation) is applied to the tissues or other target material which produces shear waves propagating in the transverse direction (e.g., perpendicular to the direction of the perturbation). The velocity at which these shear waves propagate may depend, among other parameters, on the elasticity of the tissues. For example, velocity and elasticity may be related according to the following expression: E=pv², where E is the elasticity of a tissue, p is the density of the tissue and v is the velocity of the shear wave. Therefore, by sensing the velocity at which these shear waves propagate, the elasticity may be inferred which in turn may provide an indication on the nature of the tissues. The shear waves may be monitored by imaging the target material after application of the perturbation and performing image analysis to monitor motion of the target material. In some embodiments, the imaging is ultrasound imaging performed using the same ultrasound probe(s) as used to apply the perturbation. In some embodiments, the velocity of a shear wave may be determined using time-of-flight techniques, whereby multiple images are taken and the velocity is determined based on the time it takes the shear wave to propagate across a known distance.

According to one aspect of the present application, accurate alignment of HIFU waves relative to the target tissues may be accomplished using imaging techniques based on shear waves. To that end, Applicant has appreciated that different types of tissues may have different elasticities. Therefore, the state of the tissue (e.g., whether the tissue is healthy or cancerous, and if the latter, what type of cancerous cell) being imaged may be inferred by detecting its elasticity. In this way, the region in which a particular type of tissue (e.g., carcinoma, a fibrous tissue or a cirrhosis) in need of treatment is present can be identified, thus providing guidance as to where a HIFU wave should be aimed.

Other methods for identifying and/or locating tissues in need of treatment include, but are not limited to, imaging techniques that provide contrast in the region being treated, such as x-ray computed tomography (CT), magnetic resonance imaging (MRI), positron emission tomography (PET), single-photon emission computed tomography (SPECT), and/or ultrasound imaging. In other embodiments, biopsies may be used for identifying and/or locating tissues in need of treatment.

According to another aspect of the present application, elasticity imaging techniques may be used to detect whether the HIFU waves have properly treated (or are properly treating) the target tissue, and in some embodiments, the extent to which the tissue has been treated. In particular, Applicant has appreciated that tissues that have been treated using HIFU waves may exhibit a change in elasticity relative to that of non-treated tissues. Therefore, HIFU-induced thermal lesions and/or HIFU-induced mechanical breakdown may be identified by sensing variations in elasticity, for example using elasticity imaging.

It should be appreciated that shear wave imaging is provided solely by way of example as a possible type of elasticity imaging. However, the application is not limited to shear wave imaging, as other types of elasticity may be applied. Some of these types of elasticity imaging may be used to track movements of regions of tissues, where the movement may be caused for example by pressure applied to the surface of the tissue, by contraction of muscles and/or by thumping.

The aspects and embodiments described above, as well as additional aspects and embodiments, are described further below. These aspects and/or embodiments may be used individually, all together, or in any combination of two or more, as the application is not limited in this respect.

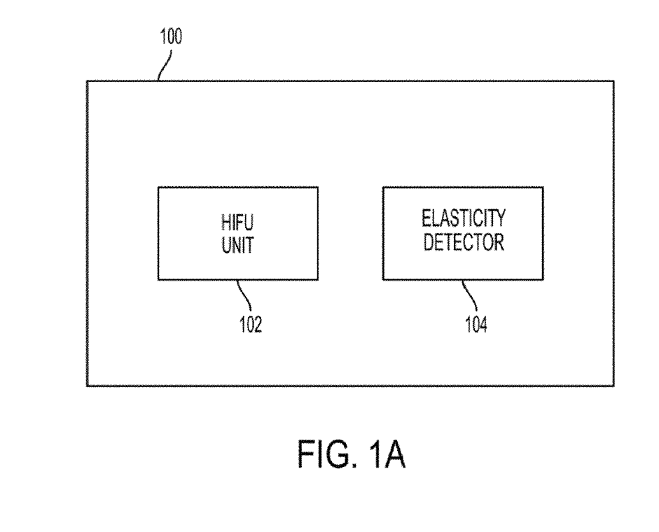

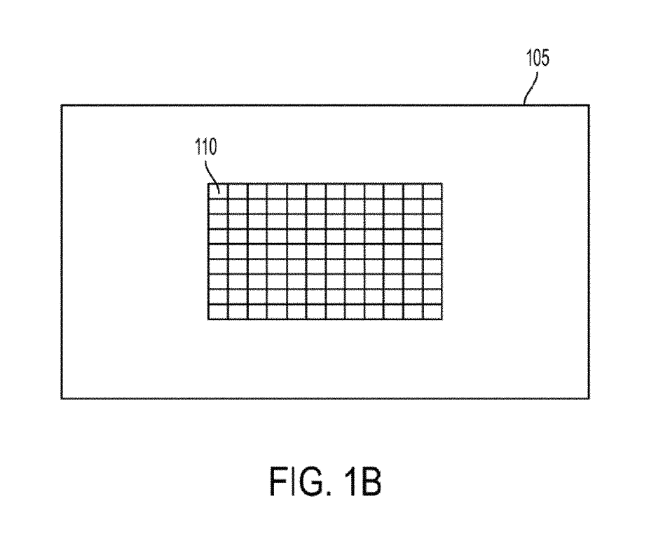

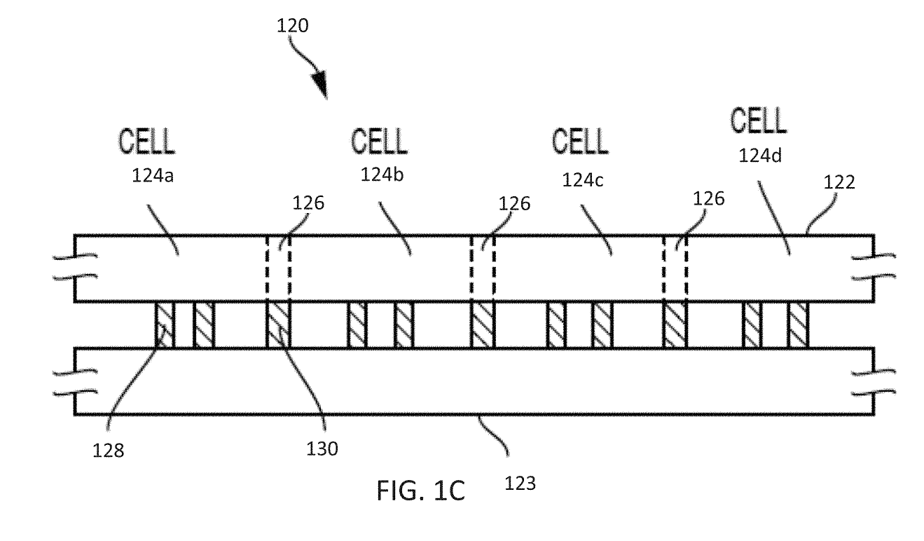

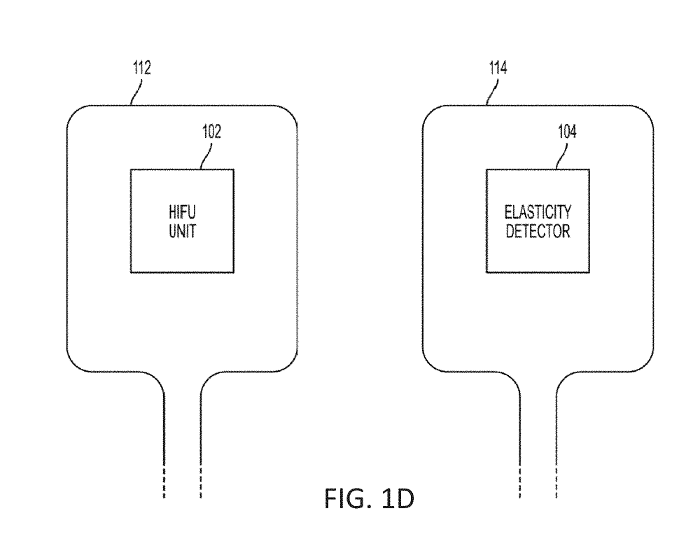

FIG. 1A is a block diagram illustrating schematically a system for treating medical conditions, such as cancers, cataracts, kidney stones, or other diseases. It should be appreciated, however, that the various aspects described herein are not limited to treating those items listed, but rather that the application of HIFU and elasticity imaging may be applied in a variety of settings for a variety of purposes. As illustrated, system 100 comprises HIFU unit 102 and elasticity detector 104. HIFU unit 102 may comprise a plurality of ultrasound elements adapted to emit and/or receive ultrasound waves. As such, each ultrasound element may operate as a source and/or a sensor. In some embodiments, these elements may be arranged as two-dimensional arrays. However, not all ultrasound elements are limited in this respect as some ultrasound elements may be arranged sparsely or irregularly.

HIFU unit 102 may be configured to emit intensities that are sufficiently large to treat medical conditions (for example through ablation). In some embodiments, HIFU unit 102 may be configured to emit intensities that are between 500 W/cm² and 20 KW/cm², between 1 KW/cm² and 20 KW/cm², between 1 KW/cm² and 10 KW/cm², between 1 KW/cm² and 9 KW/cm², between 1 KW/cm² and 7 KW/cm², between 1 KW/cm² and 5 KW/cm², between 1 KW/cm² and 3 KW/cm², between 3 KW/cm² and 10 KW/cm², or within any range within such ranges.

For comparison, the intensities emitted for imaging purposes, whether emitted by elasticity detector 104 or HIFU unit 102 may be between 100 mW/cm² and 100 W/cm², between 500 mW/cm² and 100 W/cm², between 1 W/cm² and 100 W/cm², or within any range within such ranges.

Non-limiting examples of ultrasound elements which may be used in any of the embodiments described herein include capacitive micromachined ultrasound transducers (CMUT), piezoelectric transducers, lead zirconate titanate (PZT) elements, lead magnesium niobate-lead titanate (PMN-PT) elements, polyvinylidene difluoride (PVDF) elements, high power (“hard”) ceramics such as those designated as PZT-4 ceramics, or any other suitable elements. In at least some of the embodiments in which the ultrasound elements are implemented using CMUTs, the CMUTs may be disposed on a common semiconductor substrate, such as a silicon substrate. For example, an ultrasound-on-a-chip device may be employed having a plurality of microfabricated ultrasonic transducers integrated on a substrate with integrated circuitry which controls, at least in part, application of HIFU and/or elasticity imaging.

HIFU unit 102 may be configured to perform a variety of medical treatments. Examples of medical treatments that may be performed using HIFU unit 102 include, but are not limited to, thermal ablation, histotripsy, and boiling histotripsy. When used in thermal ablation, HIFU waves may be focused on a particular tissue, such as a cancerous cell. In some embodiments, HIFU may be applied to raise the temperature of the target tissue to 42° C.−45° C., which is the temperature range to which certain cancerous cells are more sensitive than healthy cells. Of course, not all ablation treatments are limited to this range. Temperatures above 47° C. for 30-60 minutes may lead to cytotoxic effects.

In one specific example, a thermal therapy may be performed in which tissues are heated to 56° C. for about one second (e.g., between 0.5 and 1.5 seconds or between 0.25 and 5 seconds). While such a high temperature may be toxic to most cells, diffusion outside the target area may be limited due to the short period over which the treatment is performed. Such high temperatures may cause a change in material elasticity due to denaturing of the tissue.

When used in histotripsy, HIFU waves or other high intensity acoustic waves may be used to cause mechanical fractionation of tissues. Tissues treated using histotripsy may be fragmented to subcellular level. In some embodiments, histotripsy may be achieved by applying short high-intensity acoustic pulses (e.g., with a duration between 1 μs and 50 μs, a repetition rate between 10 Hz and 10 KHz, and a pressure between 5 MPa and 80 MPa). When used in boiling histotripsy, HIFU waves may be configured to cause heating shocks with highly localized temperature increases (e.g., in regions having diameters as small as 0.2 mm or less) sufficient to cause a phase change to a gas or to boil a liquid.

Elasticity detector 104 may comprise means for forming an acoustic wave, such as an ultrasound wave, which may be used to produce mechanical vibrations on a surface of a human body. In one example, elasticity detector 104 may comprise an array of ultrasound elements, such as CMUTs, for producing ultrasound waves. In some embodiments, elasticity detector 104 may further comprise means for imaging shear waves. For example, elasticity detector 104 may comprise means for sensing the velocity at which a shear wave propagates through a tissue. In some embodiments, the velocity of a shear wave may be sensed using ultrasound imaging techniques. An array of ultrasound elements, such as CMUTs, may be used at least in some embodiments to image the tissues on which the shear waves propagate.

In some embodiments, HIFU unit 102 may be used to produce acoustic waves for use in elasticity imaging. For example, acoustic waves generated by HIFU unit 102 may be aimed at a target area, and may produce mechanical vibrations through a surface of a human body. Elasticity detector 104 may be used to sense these vibrations, for example by sensing the velocity at which a shear wave propagates through a tissue. In at least some of the embodiments in which HIFU unit 102 is used to produce mechanical vibrations, the same HIFU unit may be used to treat medical conditions. For example, the HIFU unit may first be used to produce vibrations. Then, elasticity detector 104 may be used to sense these vibrations. Subsequently, HIFU unit 102 may be used to treat at least some of the tissues.

HIFU unit 102 and/or elasticity detector 104 may be implemented as an ultrasound device comprising a plurality of ultrasound elements adapted to emit and/or receive ultrasound waves. As such, each ultrasound element may operate as a source and/or a sensor. In some embodiments, these elements may be arranged as two-dimensional arrays (see for example ultrasound device 105 in FIG. 1B, which includes ultrasound elements 110). However, not all ultrasound devices 104 are limited in this respect as some ultrasound elements may be arranged sparsely or irregularly. Specific examples of ultrasound devices that may be used to implement HIFU unit 102 and/or elasticity detector 104 are described in U.S. patent application Ser. No. 15/626,330, entitled “ELECTRICAL CONTACT ARRANGEMENT FOR MICROFABRICATED ULTRASONIC TRANSDUCER,” filed on Jun. 19, 2017 and published as U.S. Pat. Pub. 2017/0365774 A1, which is hereby incorporated herein by reference in its entirety. A specific implementation of an ultrasound device that may be used in HIFU unit 102 and/or in elasticity detector 104 is illustrated in FIG. 1C, in accordance with some embodiments. The ultrasound device 120 of FIG. 1C includes an ultrasonic transducer substrate 122 bonded with an integrated circuit (IC) substrate 123. It should be appreciated that ultrasonic transducer substrates of the types described herein are not limited to being bonded with IC substrates, as they may be bonded with any other type of electrical substrate. The substrates may be wafers, and the figure illustrates a part of each substrate, as can be appreciated from the broken boundary lines. The ultrasonic transducer substrate 122 includes a plurality of ultrasonic transducers, also referred to herein as “ultrasonic transducer cells” or simply “cells” 124a, 124b, 124c, 124d, etc. In practice, a large number of such cells may be provided, such as hundreds, thousands, tens of thousands, or millions, and the various aspect of the application are not limited in this respect. Four ultrasonic transducer cells are shown for simplicity. Ultrasonic transducer cells may be electrically grouped to form an “ultrasound element.” That is, an ultrasound element may include two or more ultrasonic transducers electrically coupled to effectively operate as a single larger ultrasonic transducer. The ultrasonic transducer cells 124a-124d may each include a capacitive ultrasonic transducer, such as a CMUT. In addition, there may be an acoustic dead space 126 between at least some of the ultrasonic transducer cells. As an example, each of the cells 124a-124d may include an electrically conductive portion, for instance a bottom electrode, corresponding to a cavity of the cell. The dead space 126 may represent a portion of the same material forming the electrode, but not aligned with the cavity of the cell, and thus substantially not involved in the transduction of the cell. In some cases this acoustic “dead space” is separated from the transducer cavity by a filled trench such that the dead space is mechanically and electrically isolated from the transducer cell.

As shown, the ultrasound device 120 includes multiple, distinct physical and electrical contacts 128 between the ultrasonic transducer cells and the IC substrate 123. These contacts may be electrically conductive, and may represent bond points between the ultrasonic transducer substrate 122 and the IC substrate 123. Although two contacts 128 are shown for each of the cells 124a-124d in the exemplary embodiment depicted, it will be appreciated that other numbers are possible and it is not necessary that the same number of contacts be provided between each cell and the IC substrate. In some embodiments three contacts may be provided between an ultrasonic transducer cell and the IC substrate. FIG. 1C is a cross-sectional view, and thus additional contacts 128 may be provided in a plane closer than or farther than the plane of the page, as a non-limiting example.

The ultrasound device 120 also includes contacts 130 between the dead space 126 and the IC substrate 123. The contacts 130 may be electrically conductive and may represent bond points between the ultrasonic transducer substrate 122 and the IC substrate 123. Multiple contacts 130 may be provided between a dead space region and the IC substrate 123. IC substrate 123 may include electronic circuitry for driving the cells 124a-124d to generate acoustic waves and/or for processing signals sensed by the cells in response to receiving acoustic waves. In some embodiments, elasticity detector 104 may be disposed within IC substrate 123.

In some embodiments, HIFU unit 102 and/or elasticity detector 104 may include large densities of ultrasound devices of the types described herein. Having large densities of ultrasound devices may be useful for example to support 3D ultrasound imaging. In one example, HIFU unit 102 and/or elasticity detector 104 may include between 4000 ultrasound devices per steradian and 80000 ultrasound devices per steradian, between 6000 ultrasound devices per steradian and 80000 ultrasound devices per steradian, between 8000 ultrasound devices per steradian and 80000 ultrasound devices per steradian, between 10000 ultrasound devices per steradian and 80000 ultrasound devices per steradian, or between 12000 ultrasound devices per steradian and 80000 ultrasound devices per steradian.

In another example, HIFU unit 102 and/or elasticity detector 104 may include between 50 ultrasound devices per cm² and 1000 ultrasound devices per cm², between 100 ultrasound devices per cm² and 1000 ultrasound devices per cm², between 200 ultrasound devices per cm² and 1000 ultrasound devices per cm², or between 300 ultrasound devices per cm² and 1000 ultrasound devices per cm². The overall area of the ultrasound device array may be between 1 cm² and 400 cm², between 10 cm² and 400 cm², between 1 cm² and 10 cm², between 2 cm² and 6 cm², or between 14 cm² and 18 cm². For example, the overall area of the ultrasound device array may be 1 cm², 16 cm² or 54 cm².

HIFU unit 102 and elasticity detector 104 may be disposed on a common ultrasound device, such as a common substrate (e.g., a silicon substrate), a common support (e.g., a printed circuit board), or a common housing (e.g., a handheld probe). Alternatively, HIFU unit 102 and elasticity detector 104 may be packaged separately. FIG. 1D illustrates an example in which HIFU unit 102 is part of a first handheld probe 112 and elasticity detector 104 is part of a second handheld probe 114. The handheld probes may be used in connection with one another during performance of a medical treatment. For example, handheld probe 114 may be used to locate tissue in need of medical treatment and/or to identify tissue that has been treated. Handheld probe 112 may be used to perform the medical treatment.

While the handheld probes 112 and 114 are non-limiting examples of ultrasound devices which may be used to apply HIFU and elasticity imaging, other implementations are possible. For example, in some embodiments two or more ultrasound probes are mechanically mounted to one or more support structures. The ultrasound probes may include ultrasound-on-a-chip devices including microfabricated ultrasonic transducers with (at least some) analog and/or digital control circuitry. At least one of the ultrasound probes may be a HIFU-on-a-chip probe. The support structures may include a plate, mounting ring, bar, or other support structure, and may be mechanically adjustable. The ultrasound probes of the arrangement may include dedicated HIFU and elasticity imaging probes, or may include at least one probe which performs both elasticity imaging and HIFU. For example, one or more of the ultrasound probes of the arrangement may include an arrangement (e.g., an array) of microfabricated transducers electronically controllable to produce an ultrasound signal for HIFU or elasticity imaging. Different transducers of the ultrasound probe may be used for HIFU and elasticity imaging in some embodiments. Alternatively, the same transducers of the ultrasound probe may be used for HIFU and elasticity imaging, being operated accordingly at different times during the HIFU procedure.

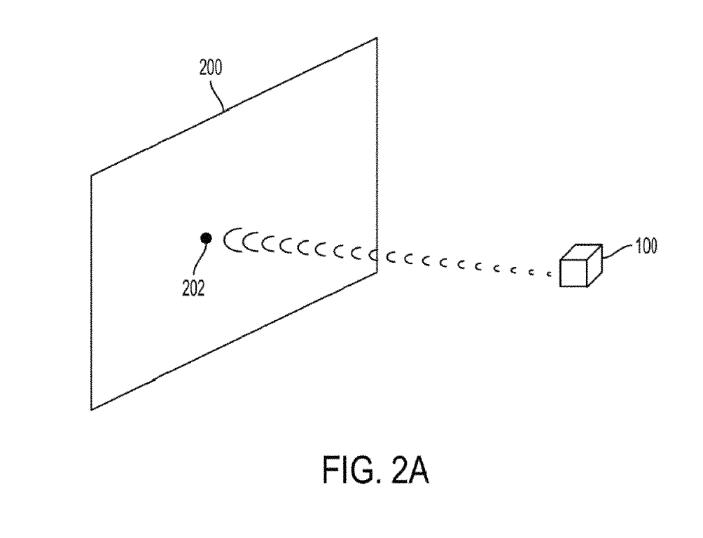

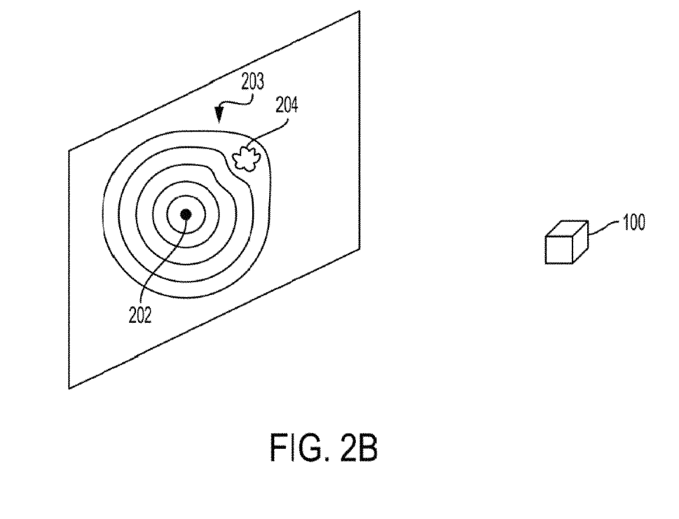

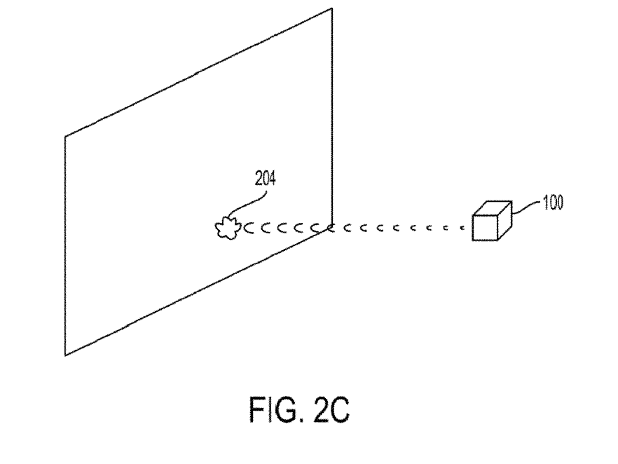

FIGS. 2A-2C are schematic diagrams illustrating how an ultrasound system, such as system 100, may be used, at least in some embodiments, to treat medical conditions. Initially, ultrasound elasticity imaging may be used to locate the target area for treatment. In the act illustrated in FIG. 2A, system 100 may emit an acoustic wave (e.g., an ultrasound wave) towards a tissue 200 of a human body. The acoustic wave may hit a region 202 of tissue 200 (e.g., at least a portion of an acoustic wavefront reaches region 202). In some embodiments, the acoustic wave can be focused on region 202, where the region 202 can have an area of less than a few square millimeters (e.g., 1-5 mm² or any other dimensions).

When region 202 is hit, the acoustic wave may cause a mechanical perturbation in the tissue 200, which may result in the generation of one or more shear waves as illustrated in FIG. 2B. The shear waves may propagate away from the impacted region. The shear waves may give rise to oscillation of the particles in the tissue that is transverse with respect to the propagation of the acoustic wave. The velocity at which the shear waves propagate may depend, among other parameters, on the elasticity of the tissue.

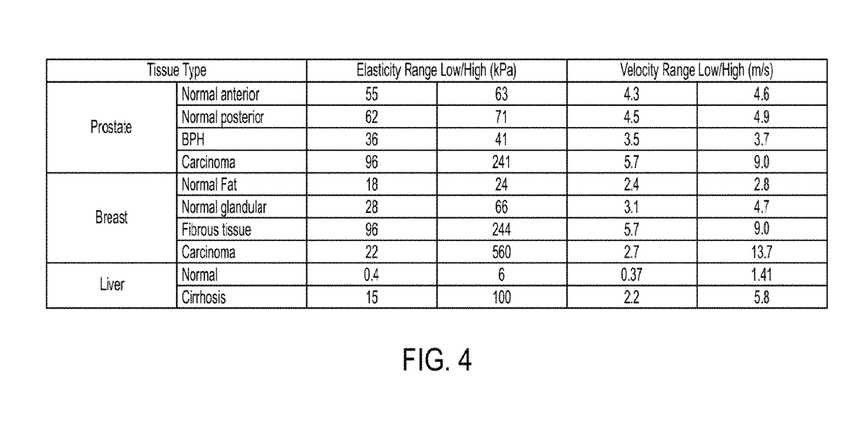

In the example of FIG. 2B, the shear wave encounters a cancerous cell region 204. Cancerous cell region 204 may have an elasticity that is different than that of the surrounding tissues. Certain cancerous cells for example are significantly stiffer than healthy tissues (as for example is shown in FIG. 4). As a result, the velocity of the shear wave varies as it propagates across the cancerous cell region 204. In some embodiments, a 3D or 2D stiffness map of tissue 200 may be obtained by sensing the local velocity of the shear wave. This may be achieved, at least in some embodiments, using Doppler imaging techniques, such as ultrasound Doppler imaging. In some embodiments, pulsed-wave Doppler imaging may be used to track the velocity of a shear wave. In some embodiments, multiple ultrasound images of the region are captured and analyzed to assess movement of the imaged structures. In some embodiments, it may be determined that a cell is in need of treatment if its elasticity is within a range deemed at risk.

Additionally, or alternatively, motion of the individual particles at the focus in region 202 generated by the HIFU beam can be directly measured by cross-correlation techniques. This movement can be detected by using a lower frequency modulation on the HIFU beam that enables detection of that modulation signal on the particle perturbation by the acoustic wave. This particle motion is related to the HIFU pressure and elasticity of the cell region, thus allowing an at-focus elasticity measurement. This method may affect only the region immediately proximate the point of focus, without affecting the surrounding tissue. This approach may enhance monitoring of the cells receiving HIFU treatment. At low average powers, this approach may improve targeting via a 3D scan of the tissue by moving the HIFU focus through various points, and measuring dynamic particle movement at the focus. This approach may allow monitoring at each application point during the treatment of the cell region itself.

In some embodiments, the velocity of the shear waves may be in the 1 m/s-10 m/s range, although other ranges are possible. As such, images having sub-millimeter resolutions may call for thousands of frames per second to detect tissue movement correlated to the shear wave propagation. To achieve shear wave velocity measurements from these detections, cross correlation techniques may be used in some embodiments. In some embodiments, shear imaging techniques may be used to achieve such large frame rates. In some embodiments, the shear waves may propagate at velocities sufficiently large to enable speckle tracking techniques, in which full movies of the shear wave propagation through the tissue may be provided. In some embodiments, a speckle tracking technique involves forming an image (or sub-image) and correlating a 2D map of the speckle with another 2D map of the speckle occurring at a different time. When speckle-based correlation techniques are used, variations in the content between frames (e.g., consecutive frames or frames separated by a certain interval) may be identified. The velocity of a shear wave may be inferred by performing a cross correlation on the data representing these variations of content. It should be appreciated that these variations may be computed by comparing entire image frames, or just by comparing sub-regions of the images, such as specific scan lines.

In some embodiments, a data ensemble is collected and a high-pass filter is used to remove static tissue signals. A data ensemble may comprise a set of measurements obtained over sufficiently short time intervals (e.g., less than 10 μs or less than 1 μs) such that positional movements in the tissues that have been displaced by shear waves can be determined. As such, in some embodiments, elasticity detector 104 may comprise circuitry for generating, collecting and/or correlating a data ensemble of the types described herein to perform shear wave tracking measurements. In some embodiments, the high-pass filter may be part of elasticity detector 104, and may be coupled to the receiving ultrasound devices.

In some embodiments, the relative phase difference between measurements may provide an indication as to the position shift (e.g., the movement of a tissue displaced by shear waves) induced by the shear wave. For example, a position shift equal to the wavelength of the acoustic wave may be inferred from a 2n-phase difference. The position of these scattering tissue (also referred to as “scatterers”) may change as a shear wave propagates through the tissue. The elasticity detector 104 can detect the displacement of the scatterers in the tissues as a shear wave is traveling through the tissue.

In one example, tissue-level Doppler techniques may be used to track the velocity of a shear wave. In this example, shear wave scattered from tissues may be tracked. Shear waves scattered from tissues may be discerned from shear waves scattered from blood cells based on the amplitude of the waves. Discerning these types of scattered shear waves from one another may be performed using filters, including for example wall filters. In some circumstances, the shear waves scattered from tissues may have intensities several dB higher than those scattered from blood cells, such as between 10 dB and 60 dB higher, between 10 dB and 50 dB higher, between 10 dB and 40 dB higher, between 10 dB and 30 dB higher, between 10 dB and 20 dB higher, or within any range within such ranges.

In some embodiments, a lag-1 autocorrelation is used to estimate shear wave velocities. In some embodiments, Fourier filtering is used to isolate shear wave component directions by isolating effects of a shear wave propagating along a specific direction. In this manner, the time-of-flight (and hence the velocity) through a tissue along a specific direction may be estimated.

In some embodiments, a calibration procedure may be used to ensure that the beams emitted by system 100 are focused on the target region. Accordingly, some calibration procedures may be employed to determine the position of system 100 relative to the target region. For example, a suitable position for the system 100 may be determined by precisely estimating the speed of sound of a region of a tissue. In some embodiments, the speed of sound through the tissues may be inferred from the elasticity of the tissues (which for instance may be calculated using elasticity imaging). The speed of sound may be used, at least in some embodiments, to estimate, at least in part, the time-of-flight of an ultrasound beam between system 100 and the target area, which may in turn be used to improve the positioning of system 100.

Once cancerous cell region 204 (or other types of tissues) has been located, HIFU techniques of the types described above may be used as shown in FIG. 2C. HIFU techniques may be used to induce a temperature and/or mechanical change in a tissue or a cell of a subject. In some embodiments, HIFU waves may be aimed at the cancerous cell region 204 so that the cancerous cell experiences a temperature rise (for example in the 42° C.−45° C. range or above 45° C.). Other medical treatment techniques such as histotripsy or boiling histotripsy may alternatively or additionally be used. The application of HIFU may be applied by a distinct ultrasound device dedicated to HIFU in some embodiments. In other embodiments, as described above, the HIFU may be applied by an ultrasound device (e.g., an ultrasound probe) which performed the elasticity imaging.

Alternatively, or additionally, HIFU elements may be used to cause a change in a mechanical property of a tissue or cell. For example, when used in micro-cavitation, HIFU may induce a shock wave at the target area (e.g., at the focal plane of the HIFU). Micro-cavitation may be enabled by applying short HIFU pulses (e.g., between 1 μs and 10 μs) to cause waves of large pressures (e.g., between 5 MPa and 80 MPa). In some embodiments, when short HIFU pulses are applied to a tissue, a vapor cavity or a liquid-free zone (e.g., a bubble) may be formed. A shock wave may be generated when the vapor cavity or liquid-free zone implodes. In some embodiments, bubbles may be formed such that the target region is between bubbles. In some embodiments, the bubbles exhibit high reflectance, which may induce multiple scattering and thus multipath absorption in the tissue.

Alternatively, or additionally, HIFU may be used to perform ablation. Ablation may be performed, at least in some embodiments, once a multipath absorption has been created, for example via micro-cavitation. In this way, the energy needed to perform ablation may be substantially reduced. Furthermore, in this way, the energy outside the target region may be reduced, thus, limiting damage to healthy tissues located nearby.

In some embodiments, the state of the target cell (cancerous cell region 204 in this example) may be monitored throughout the application of the HIFU procedure. For example, thermal lesions caused by HIFU exhibit increased stiffness relative to non-treated tissues, and as such monitoring of the state of the target cell may be performed using shear waves in some embodiments. In some embodiments, the procedure may continue until it is determined that a characteristic of the target cells has reached a safe level (for example, when tissue's stiffness is within a certain range). This may be accomplished, at least in some embodiments, using ultrasound signals to infer tissue characteristics (e.g., stiffness, presence of bubbles, temperature). In some embodiments, the status of micro-cavitation may be monitored by sensing the intensity of backscattered ultrasounds. Accordingly, the backscattered ultrasounds from bubbles may be significantly larger relative to non-treated tissues. The monitoring may be performed using an ultrasound imaging probe in some embodiments. In some embodiments, the monitoring is performed using the same ultrasound probe(s) used to apply the HIFU. Monitoring application of the HIFU may provide verification of the intended treatment outcome. In some embodiments, the state of untreated cells may be monitored to determine whether these cells have been accidentally damaged as a result of the HIFU application. Techniques similar to those described in connection with monitoring of target cells may be used.

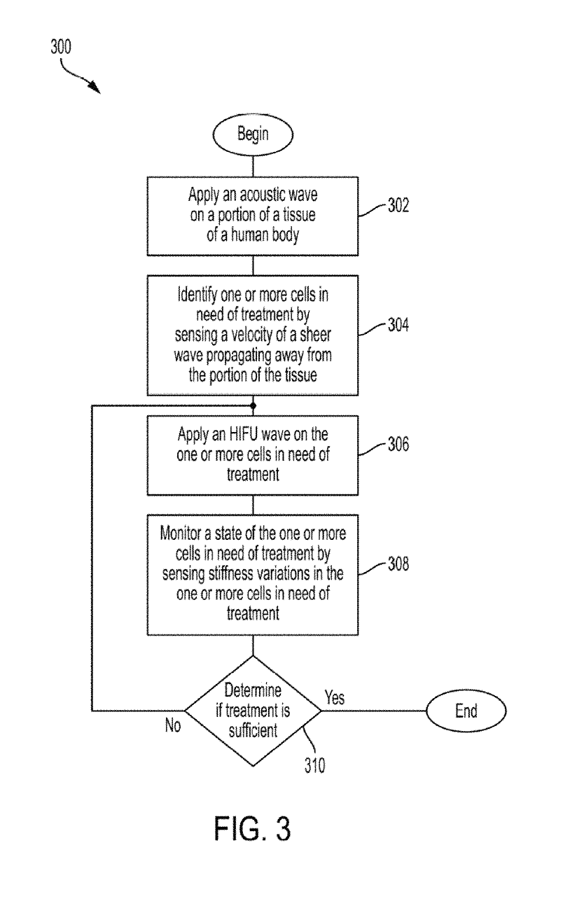

A representative method for treating medical conditions according to some embodiments of the present application is depicted in FIG. 3. Representative method 300 begins at act 302, in which an acoustic wave (e.g., an ultrasound wave) is applied to a portion of tissue of a human body (or an animal or other subject). In act 304, one or more cells in need of treatment may be identified by sensing the velocity of a shear wave propagating away from the portion of the tissue. The shear wave may arise in response to the acoustic wave hitting the portion of the tissue. Sensing of the shear wave's velocity may be accomplished using a elasticity detector of the types described above. In act 306, the medical condition may be treated by applying an HIFU wave on the identified cell region(s). The HIFU wave may for example cause a temperature change and/or a mechanical fractionation of the cell region(s). In some embodiments, micro-cavitation is performed using HIFU and subsequently ablation is performed via HIFU. Of course, ablation may be performed, in other embodiments, without first performing micro-cavitation. Optionally, in act 308, the state (e.g., whether the tissue is healthy or cancerous, and if the latter, what type of cancerous cell) of the treated cell region(s) may be monitored by sensing stiffness variations of the treated cells. The stiffness variations may be sensed for example by sensing the velocity of a shear wave produced by hitting the cell region(s) with an acoustic wave. Monitoring of the state of the treated cell region(s) may be performed while the HIFU wave is being applied, or subsequently.

In some embodiments, three dimensional (3D) imaging techniques may be used for monitoring the state of a treated regions. 3D imaging may be performed by acquiring samples on azimuth and elevation components using a 2D array of ultrasound elements. In one example, 3D imaging may be used to identify the presence of bubbles, which in some circumstances may give rise, due to their nature, to scattering with high intensity. To that end, a scattering response having a large magnitude may indicate that a bubble is present. It should be appreciated that the presence of bubbles may also be identified using triangulation techniques, at least in some embodiments. In another example, 3D imaging may be used to identify tissues that have been thermally cooked (e.g., that have experienced a temperature increase of 2° C. or more, 5° C. or more 10° C. or more, or 20° C. or more). In yet another example, 3D imaging may be used to identify the presence of hypoechoic regions (regions of poor ultrasound scattering) such as regions that have gone through mechanical ablation.

In some of the embodiments in which HIFU-based thermal procedures are performed, the time-varying temperature of a region may be tracked by comparing backscattering data (phase or magnitude of backscattered waves). For example, backscattering data between different acquisitions may be compared in some embodiments. When thermal HIFU-thermal procedures are performed, the targeted regions may contract or expand, possibly in three dimensions. By comparing backscattering data among different acquisitions, spatial variations in the targeted region due to thermal expansion or contraction may be determined. Suitable filters (e.g., wall filters) may be used to remove static components.

Optionally, in act 308, the temperature of the treated region can be monitored to determine whether the desired temperature for thermal ablation has been reached. Additionally or alternatively, the temperature of untreated tissues can be monitored to ensure that such tissues remain within a safe temperature range (e.g., within 1° C. or 2° C. of the temperature of the tissues before the treatment is applied) during the therapy. Temperature variations may for example be monitored using the ultrasound devices of HIFU unit 102 and/or elasticity detector 104, where the ultrasound devices may be configured for thermal measurements. The echo strain may be computed using a low-pass axial differentiator, which may for example be implemented using a finite-impulse response digital filter. Alternatively, or additionally, a recursive axial filter that acts as a spatial differentiator-integrator of echo shifts may be employed.

In some embodiments, speckle tracking and/or lateral shifts between frames may be used in the thermally-induced echo strain model to improve accuracy. To enhance the accuracy of speckle tracking, high sampling rate may be used. The sampling rate may be, for example, between 10 Hz and 30 KHz, between 100 Hz and 30 KHz, between 1 KHz and 30 KHz or between 10 KHz and 30 KHz. Additionally, or alternatively, correlation measurements may be performed to improve the frame rate. In this way, it may be insured that global motion of the imaged area does not affect the local temperature-induced variations.

In act 310, it may be determined whether the treatment of act 306 is sufficient. This determination may be performed in any suitable way, such as by determining whether a parameter associated with the shear wave (e.g., the velocity) or a characteristic of the tissues (e.g., elasticity, stiffness, Young's modulus, pressure measured for example in pounds per square inch (PSI) or pascals, ratio of stress to strain, or other related quantities) is within a certain range, or above or below a certain threshold. Examples of characteristics associated with medical conditions for a representative prostate, breast and liver are illustrated in FIG. 4. Of course, these characteristics are only provided by way of example. If it is determined that the treatment is sufficient, method 300 may end. Otherwise, method 300 may iterate and act 306 may continue or may be repeated. For example, if a breast exhibits an elasticity of about 20 kPa, it may be inferred that the breast is healthy, and method 300 may end. In another example, if it is determined that the velocity of a shear wave through a tissue of a liver is about 3.3 m/s, it may be inferred that a cirrhosis may be present, and the treatment may continue.

Representative method 300 may be performed using one ultrasound device (which may include a substrate, a support and/or a housing, such as a handheld probe) comprising one or more HIFU units and one or more elasticity detectors. Alternatively, multiple ultrasound devices may be used to perform representative method 300. For example, a handheld probe may be used which comprises an HIFU unit and another handheld probe may be used which comprises a elasticity detector. In some embodiments, multiple handheld probes each comprising an HIFU unit may be used to produce high intensities.

It should be appreciated that, in addition to (or in alternative to) elasticity imaging of the types described herein, other ways of inferring tissue elasticity may be used. In one example, compression measurements may be used to infer elasticity. Compression measurements may be performed by applying differing amounts of pressure to a tissue and by sensing the relative deformation of the scattering regions. In some embodiments, elasticity can be estimated by measuring correlated points in one ultrasound image that change position in subsequent ultrasound images based on the different amount of force. Different amount of force may be generated, at least in some embodiments, by varying the pressure with which a probe is placed in contact with a target area of a subject. Elasticity may be estimated in this manner in one, two or three dimensions.

What is claimed is:

1. An apparatus comprising:
one or more high intensity focused ultrasound (HIFU) units configured to generate HIFU waves and one or more elasticity detectors configured to sense a characteristic of a shear wave, the one or more HIFU units and the one or more elasticity detectors being disposed on a common ultrasound device.

2. The apparatus of claim 1, wherein the ultrasound device comprises a substrate on which ultrasonic transducers are integrated.

3. The apparatus of claim 1, wherein the ultrasound device comprises a handheld probe.

4. The apparatus of claim 1, wherein at least one of the one or more HIFU units comprises a capacitive micromachined ultrasound transducer (CMUT).

5. The apparatus of claim 1, wherein at least one of the one or more HIFU units is configured to emit an acoustic intensity that is between 500 W/cm² and 20 KW/cm².

6. The apparatus of claim 1, wherein the one or more HIFU units are disposed on a first substrate, the first substrate being bonded to a second substrate comprising electronic circuitry electrically coupled to the one or more HIFU units.

7. The apparatus of claim 1, wherein the one or more elasticity detectors are configured to sense a velocity of the shear wave.

8. A method for treating medical conditions, the method comprising:
applying an acoustic wave on a portion of a tissue of a human body;
identifying one or more cells in need of treatment by sensing a characteristic of a shear wave arising in response to the application of the acoustic wave and propagating away from the portion of the tissue; and
applying a high intensity focused ultrasound (HIFU) wave on the one or more cells in need of treatment.

9. The method of claim 8, further comprising monitoring a state of the one or more cells in need of treatment by sensing stiffness variations in the one or more cells in need of treatment.

10. The method of claim 9, wherein the acoustic wave is a first acoustic wave and the shear wave is a first shear wave, and wherein sensing stiffness variations in the one or more cells in need of treatment comprises:
applying a second acoustic wave to the one or more cells in need of treatment; and
sensing a characteristic of a second shear wave arising in response to the application of the second acoustic wave and propagating away from the one or more cells in need of treatment.

11. The method of claim 8, wherein identifying one or more cells in need of treatment by sensing a characteristic of a shear wave propagating away from the portion of the tissue comprises identifying one or more cells in need of treatment by sensing a velocity of a shear wave propagating away from the portion of the tissue.

12. The method of claim 8, wherein applying an HIFU wave on the one or more cells in need of treatment comprises emitting an acoustic intensity that is between 500 W/cm² and 20 KW/cm².

13. The method of claim 8, further comprising:
performing micro-cavitation on the one or more cells in need of treatment,
wherein applying an HIFU wave on the one or more cells in need of treatment comprises applying an HIFU wave on the micro-cavitation.

14. The method of claim 13, further comprising determining a state of the micro-cavitation by sensing a backscattered ultrasound wave.

15. The method of claim 8, further comprising identifying a type of the tissue in need of treatment based on the characteristic of the shear wave

16. A method for treating medical conditions:
applying a high intensity focused ultrasound (HIFU) wave on a portion of a tissue of a human body;
determining a state of the portion of the tissue by monitoring the portion of the tissue using ultrasound waves; and
updating the application of the HIFU wave based on the determined state of the portion of the tissue.

17. The method of claim 16, wherein monitoring the portion of the tissue comprises monitoring a shear wave propagating through the tissue.

18. The method of claim 17, wherein monitoring a shear wave propagating through the tissue comprises determining a velocity of the shear wave.

19. The method of claim 18, further comprising generating an elasticity map based on the determined velocity of the shear wave.

20. The method of claim 19, further comprising identifying a region in need of treatment based on the elasticity map, and applying the HIFU wave to the region in need of treatment.
